(19) United States
(12) Patent Application Publication
(10) Pub. No.: US 2025/0185950 A1
(43) Pub. Date: Jun. 12, 2025

(54) SYSTEM AND METHOD FOR WIRELESS TRANSMISSION OF GLUCOSE DATA

(71) Applicant: Laxmi Therapeutic Devices, Inc., Goleta, CA (US)

(72) Inventors: Timothy J. UHL, Goleta, CA (US); Shane A. WILLIAMS, Santa Barbara, CA (US); Romeo CHAHINE, Goleta, CA (US); Thomas METZMAKER, Goleta, CA (US)

(21) Appl. No.: 18/536,176

(22) Filed: Dec. 11, 2023

(51) Int. Cl. A61B 5/145; A61B 5/00; A61B 5/01; G08C 17/02

(52) U.S. Cl. CPC A61B 5/14532; A61B 5/0004; A61B 5/01; A61B 5/7275; G08C 17/02; A61B 2560/0223

(57) ABSTRACT

A system and method for wireless transmission of glucose data. According to an embodiment of the present disclosure, there is provided a system, including: a glucose sensor; a beacon transmitter; and a processing circuit connected to the glucose sensor and the beacon transmitter, the processing circuit being configured to transmit a data packet, the data packet including measurement data including a measurement value, the measurement value being based on a glucose measurement.

### FIELD

One or more aspects of embodiments according to the present disclosure relate to glucose measurements, and more particularly to a system and method for wireless transmission of glucose data.

### BACKGROUND

A continuous glucose monitor (CGM) may be an important health monitoring device in various circumstances. For example, a diabetic or pre-diabetic subject may use such a monitor to guide decisions related to meal planning, insulin dosing, or seeking emergency medical care. It may be advantageous for the continuous glucose monitor to be connected, e.g., through a wireless link, to another device, such as a mobile phone; such a connection may make it more convenient for the user to view glucose data and may make it possible for the data to be transmitted (e.g., over the internet) to a health-care provider.

It is with respect to this general technical environment that aspects of the present disclosure are related.

### SUMMARY

According to an embodiment of the present disclosure, there is provided a system, including: a glucose sensor; a beacon transmitter; and a processing circuit connected to the glucose sensor and the beacon transmitter, the processing circuit being configured to transmit a data packet, the data packet including measurement data including a measurement value, the measurement value being based on a glucose measurement.

In some embodiments, the data packet complies with standard 802.15.1 promulgated by the Institute of Electrical and Electronics Engineers.

In some embodiments, the data packet includes a packet payload including a header and a payload.

In some embodiments, the header of the packet payload includes a Protocol Data Unit field having a value of 2.

In some embodiments, the payload of the packet payload includes the measurement data.

In some embodiments, the measurement data is encrypted.

In some embodiments, the measurement data further includes a sample identifier.

In some embodiments, the measurement data further includes a measurement quality indicator.

In some embodiments, the measurement data further includes an indication of a battery voltage of the system.

In some embodiments, the glucose measurement is a measurement of a glucose level of a subject, and the measurement data further includes an indication of a skin temperature of the subject.

In some embodiments, the measurement data further includes a status indicator indicating a status of the system.

In some embodiments, the measurement data further includes a trend measurement.

In some embodiments, the measurement value includes a raw glucose measurement.

In some embodiments, the measurement value includes a calibrated glucose measurement.

In some embodiments, the system does not include a radio receiver.

### BRIEF DESCRIPTION OF THE DRAWINGS

These and other features and advantages of the present disclosure will be appreciated and understood with reference to the specification, claims, and appended drawings wherein:

FIG. 1 is a block diagram of a system for monitoring glucose levels, according to an embodiment of the present disclosure;

FIG. 2 is a block diagram of a system for monitoring glucose levels, according to an embodiment of the present disclosure; and

FIG. 3 is a block diagram of a system for monitoring glucose levels, according to an embodiment of the present disclosure.

### DETAILED DESCRIPTION

The detailed description set forth below in connection with the appended drawings is intended as a description of exemplary embodiments of a system and method for wireless transmission of glucose data provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

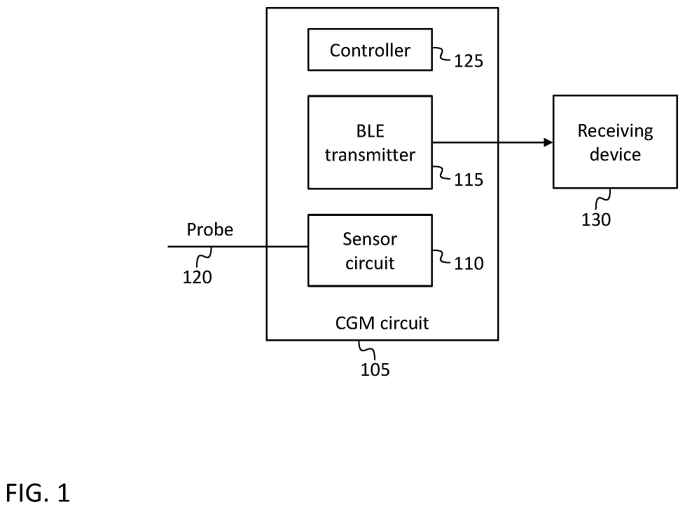

Referring to FIG. 1, in some embodiments, a continuous glucose monitoring (CGM) circuit 105 includes a sensor circuit 110 and a beacon transmitter 115. The beacon transmitter 115 may be a Bluetooth Low Energy transmitter (e.g., a transmitter configured to transmit data packets that comply with standard 802.15.1 promulgated by the Institute of Electrical and Electronics Engineers), or it may be a different wireless beacon transmitter. Such a data packet may include measurement data, which may include, e.g., (i) a measurement value (e.g., a glucose measurement), (ii) a trend measurement, and other data (discussed in further detail below), such as a sample identifier. As used herein, a “beacon transmitter” is a one-directional transmitter that does not rely on a receiver (e.g., for handshaking or for establishing a connection with another device). In some embodiments, the continuous glucose monitoring circuit 105 lacks a radio receiver, and, in some embodiments, it lacks a receiving data path generally, e.g., there is no data path into the continuous glucose monitoring circuit 105 once manufacturing of the continuous glucose monitoring circuit 105 is complete (e.g., there is no mechanism for data to be received by the continuous glucose monitoring circuit 105). Such an absence of a radio receiver may reduce power consumption, and the absence of a receiving data path may improve the security of the continuous glucose monitoring circuit 105, e.g., it may be an obstacle to malicious actors who may wish to program the continuous glucose monitoring circuit 105 with malicious code.

The sensor circuit 110 may include a probe 120 that is inserted below the skin of a user (e.g., a subject or a patient) for the purpose of sensing a glucose level (e.g., an interstitial glucose level) of the user. The probe 120 may generate a current that is affected by, and that is an indication of, a glucose level of the user. The current may be measured by the sensor circuit 110 and converted to a digital representation, which may be transmitted by the beacon transmitter 115, as part of a data packet. The continuous glucose monitoring circuit 105 may include a controller 125, which may be a processing circuit (discussed in further detail below), and which may control the operations of the continuous glucose monitoring circuit 105. The controller may be a component, of the continuous glucose monitoring circuit 105, which is connected to the sensor circuit 110 and to the beacon transmitter 115, or it may be integrated into one or both of the sensor circuit 110 and the beacon transmitter 115 (e.g., each of the sensor circuit 110 and the beacon transmitter 115 may include a respective microcontroller (and the controller 125 may be a distributed controller including both microcontrollers)).

The data packet transmitted by the beacon transmitter 115 may be received by a receiving device 130. The receiving device 130 may be a user device (e.g., a mobile telephone) in a home setting, or it may be a piece of clinical equipment, such as an integrated health monitoring station, in a clinical setting.

A combination of a glucose measurement and a trend measurement may be helpful to the user in (e.g., it may necessary to the user for) decision-making, e.g., in deciding whether to consume certain foods, in deciding whether to administer insulin, and, if so, at what dose, or in deciding whether to exercise. In some embodiments, the trend measurement is generated by the receiving device 130 from a plurality of recently-received glucose measurement (e.g., by processing these glucose measurements with a finite impulse response (FIR) filter or with an infinite impulse response (IIR) filter).

In some circumstances, a glucose measurement without a trend measurement may be insufficient for decision-making by the user. In some embodiments, therefore, a trend measurement is generated by the continuous glucose monitoring circuit 105 (e.g., by processing glucose measurements with a finite impulse response (FIR) filter or with an infinite impulse response (IIR) filter) and included in the measurement data of the data packet; this may have the advantage that when a data packet is received by the receiving device 130 after one or more packets having been missed (e.g., because the receiving device 130 was out of range), it may nonetheless be possible to provide to the user both a glucose measurement and a trend measurement, which may be sufficient for immediate decision making by the user (e.g., it may make it unnecessary for the user to wait for additional measurements to be obtained). As such, in some embodiments, each data packet transmitted by the beacon transmitter 115 may include measurement data which includes a glucose measurement and a trend measurement.

The measurement data may further include a sample number, which may be a number that is initialized (e.g., to zero or one) when the continuous glucose monitoring circuit 105 first begins operating (e.g., when the continuous glucose monitoring circuit 105 is first powered up, or when the sensor circuit 110 is first connected to the other components of the continuous glucose monitoring circuit 105, in an embodiment in which the parts are connected together after (or just before) the probe is inserted into the user.

In operation, the sensor circuit 110 may perform a series of periodic glucose measurements, each resulting in a respective raw glucose measurement. A calibration (e.g., sensitivity scaling or offset adjustment) may then be applied to each raw glucose measurement to generate a calibrated glucose measurement. The calibration may involve correcting for factors (which may be referred to as “sensor-specific calibration factors”) that vary between sensor circuits 110 during manufacture, or correcting for factors (which may be referred to as “subject-specific calibration factors”) that vary depending on how (e.g., to what depth) the probe is inserted into the subject. Sensor-specific calibration factors may be measured at the time of manufacture, using in-vitro testing of each sensor, or of one or more sensors from each batch of sensors, and they may be saved in the continuous glucose monitoring circuit 105 (e.g., in the sensor circuit 110). Subject-specific calibration factors may be measured using a finger-stick reference measurement after the probe has been inserted into the patient. In some embodiments, the subject-specific calibration factors are also saved in the glucose monitoring circuit 105. In such an embodiment, a measurement value of each data packet may be a calibrated glucose measurement.

In some embodiments, insufficient calibration data may be saved in the continuous glucose monitoring circuit 105 to generate a calibrated glucose measurement from a raw glucose measurement. For example, if there is no data path into the continuous glucose monitoring circuit 105 once manufacturing is complete, it may not be feasible to save subject-specific calibration factors in the continuous glucose monitoring circuit 105. In such an embodiment, each measurement value may be (i) a partially-calibrated glucose measurement (e.g., one that is calibrated only with respect to sensor-specific calibration factors) or a (ii) raw glucose measurement, and the receiving device 130 may convert the received measurement value into a calibrated glucose measurement.

In some embodiments, e.g., if the beacon transmitter 115 is a Bluetooth Low Energy transmitter, each data packet may include (i) a preamble, (ii) an access address, (iii) a packet payload, and (iv) a cyclic redundancy check (CRC). The packet payload may include a header and a payload (the payload of the packet payload may be referred to simply as the “payload”, for brevity). The header of the packet payload may include several fields including a Protocol Data Unit (PDU) field, which may be set to 2 (binary 0010) to signal that the PDU type is a Non-connectable undirected advertising event.

The payload may include the measurement data, which may include one or more of the following eights fields.

1. A unique device identifier.

2. A sample identifier, e.g., a sample number.

3. A piece of (calibrated or uncalibrated) measurement data.

4. Sample quality information (or a “measurement quality indicator”). This may be a bitfield that represents confidence in the sample. Examples of quality information may include: whether voltages or currents that were outside of corresponding specified acceptable ranges were detected during a measurement, whether excessively rapid signal changes were detected during a measurement, whether the signal variance within a measurement window exceeded a specified maximum acceptable value, and whether the sensor is present in an incorrect tissue space.

5. Trend Information. Trend information may include discrete-valued trend indicators or one or more quantitative trend measurements quantifying the rate at which samples are changing over time. A discrete-valued trend indicator may indicate whether the glucose measurement is rising, falling, rapidly rising, or rapidly falling.

6. Battery Voltage.

7. Skin Temperature.

8. Device Status. Examples of device status indications include transmitter over-temperature, sensor activation failed, sensor warmup in progress, and sensor warmup complete.

The measurement data (e.g., the payload of the packet payload) may be encrypted to help ensure the privacy of the measurement data (e.g., to prevent unauthorized receivers from recovering the glucose measurements), and decrypted by the receiving device 130. Suitable encryption and decryption keys may be stored in the continuous glucose monitoring circuit 105 and in the receiving device 130, respectively, using methods discussed in further detail below.

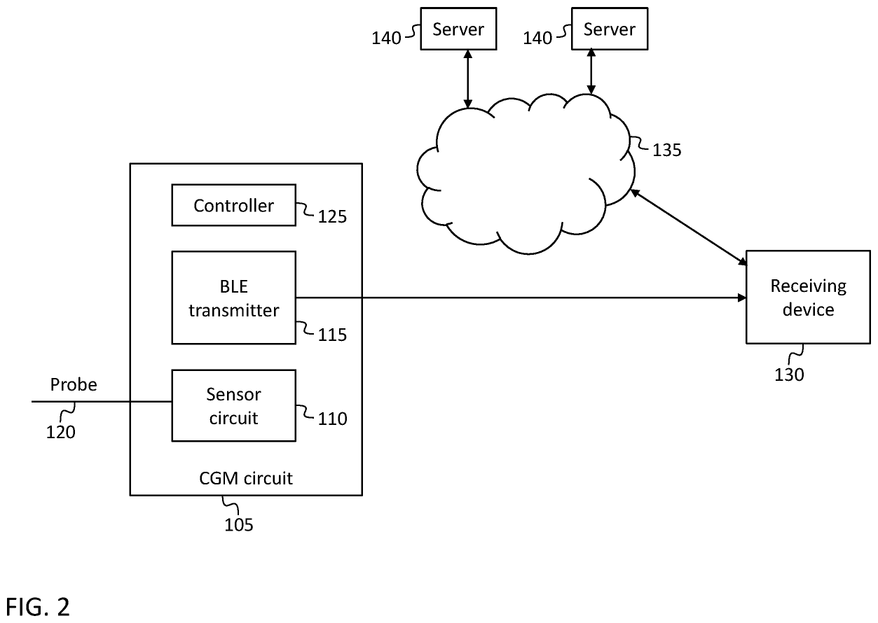

Upon receiving a data packet, the receiving device 130 may decrypt the payload, apply a calibration and display the calibrated glucose measurement as well as the trend measurement to the user. Referring to FIG. 2, it may also store the data locally, or forward it (e.g., via the internet, or “cloud” 135) to one or more other recipients, e.g., server 140, which may host a health care provider database. It may also display to the user information based on (i) the sample number (e.g., an indication of whether any data packets have been missed), (ii) the sample quality information, (iii) the battery voltage, (iv) the skin temperature, and (v) the status of the continuous glucose monitoring circuit 105.

The continuous glucose monitoring circuit 105 may not be aware of absolute time. As such, the time associated with a measurement value may be determined by the receiving device 130, e.g., using a real time clock within the receiving device 130. The receiving device 130 may also determine whether to issue a patient-specific high glucose or low glucose alarm, a rate of increase alert, or a rate of decrease alert.

As mentioned above, the payload of the packet payload may be encrypted. To this end, the continuous glucose monitoring circuit 105 may have stored in it (at the time of manufacture) an encryption key, and the receiving device 130 may have a corresponding decryption key. The corresponding decryption key may be stored, for example, in the package in which the continuous glucose monitoring circuit 105 is sold, in a manner that prevents it from being read unless a seal in or on the package is first broken (e.g., the decryption key may be printed on a card and covered with an opaque coating that may be scratched off by the user). In such an embodiment, the decryption key may, for example, be keyed into the receiving device 130 by the user. In other embodiments the receiving device may receive the decryption key via a secure internet connection. For example, when the user orders a continuous glucose monitoring circuit 105 for the first time, the user may set up an account with the vendor of the continuous glucose monitoring circuit 105 (e.g., by connecting to a server 140 operated by the vendor), and install an application, supplied by the vendor, on the user's mobile telephone. A subsequent order of another receiving device 130 by the same user (e.g., when the battery of the continuous glucose monitoring circuit 105 the user is using begins to run low) may be associated with the user's account and a server 140 operated by the vendor may, in response to the new order, automatically communicate the new decryption key to the receiving device 130 (e.g., through the application).

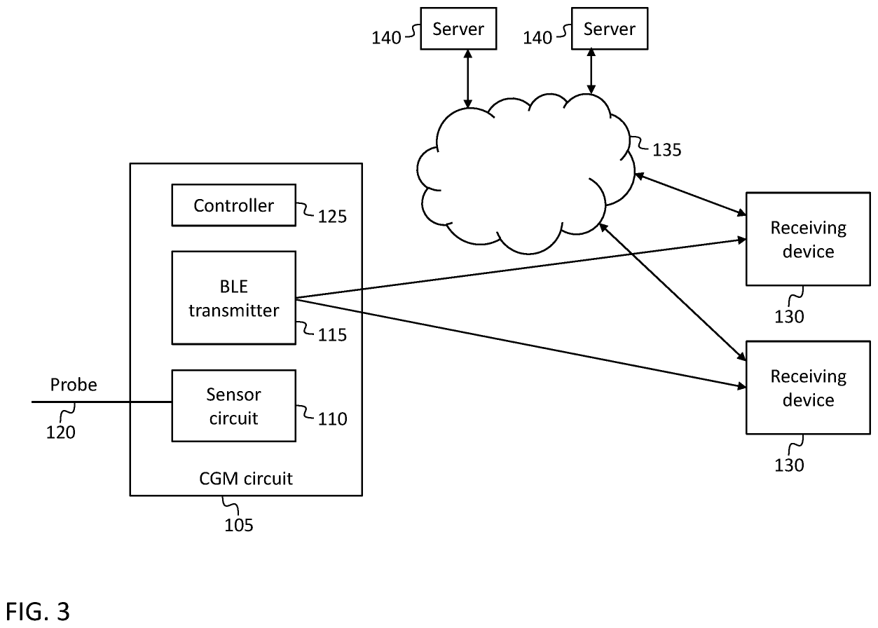

Referring to FIG. 3, in some embodiments, multiple receiving devices 125 may be associated with a user's account and may similarly receive the decryption password automatically. For example, a mobile telephone of a family member of the user may also have the application installed on it and may have the decryption password, so that the family member is able to view the user's glucose levels and trends, and so that the family member's mobile telephone may operate as a backup receiving device 130 if, for example, the user's device is misplaced or runs out of charge.

In some embodiments, the continuous glucose monitoring circuit 105 includes a Near-Field Communication (NFC) receiver, through which it may receive commands and data. For example, the NFC receiver may be used to store calibration factors in the continuous glucose monitoring circuit 105, so that it may be capable of generating calibrated glucose measurements and calibrated trend measurements. As another example, the continuous glucose monitoring circuit 105 may receive, through the NFC receiver, a command to re-transmit some number of previously obtained glucose measurements (which may be archived in the continuous glucose monitoring circuit 105); this may be useful if a number of measurements have been missed, e.g., because the continuous glucose monitoring circuit 105 was not within range of a receiving device 130.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1-35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for wireless transmission of glucose data have been specifically described and illustrated herein, many modifications and variations will be apparent to those skilled in the art. Accordingly, it is to be understood that a system and method for wireless transmission of glucose data constructed according to principles of this disclosure may be embodied other than as specifically described herein. The invention is also defined in the following claims, and equivalents thereof.

What is claimed is:

1. A system, comprising:
a glucose sensor;
a beacon transmitter; and
a processing circuit connected to the glucose sensor and the beacon transmitter, the processing circuit being configured to transmit a data packet, the data packet comprising measurement data comprising a measurement value, the measurement value being based on a glucose measurement.

2. The system of claim 1, wherein the data packet complies with standard 802.15.1 promulgated by the Institute of Electrical and Electronics Engineers.

3. The system of claim 2, wherein the data packet comprises a packet payload comprising a header and a payload.

4. The system of claim 3, wherein the header of the packet payload comprises a Protocol Data Unit field having a value of 2.

5. The system of claim 3, wherein the payload of the packet payload comprises the measurement data.

6. The system of claim 1, wherein the measurement data is encrypted.

7. The system of claim 1, wherein the measurement data further comprises a sample identifier.

8. The system of claim 1, wherein the measurement data further comprises a measurement quality indicator.

9. The system of claim 1, wherein the measurement data further comprises an indication of a battery voltage of the system.

10. The system of claim 1, wherein the glucose measurement is a measurement of a glucose level of a subject, and the measurement data further comprises an indication of a skin temperature of the subject.

11. The system of claim 1, wherein the measurement data further comprises a status indicator indicating a status of the system.

12. The system of claim 1, wherein the measurement data further comprises a trend measurement.

13. The system of claim 1, wherein the measurement value comprises a raw glucose measurement.

14. The system of claim 1, wherein the measurement value comprises a calibrated glucose measurement.

15. The system of claim 1, not comprising a radio receiver.

16. The system of claim 1, wherein the measurement data is encrypted.
